(19) United States
(12) Patent Application Publication
(10) Pub. No.: US 2010/0256771 A1
(43) Pub. Date: Oct. 7, 2010

(54) ACETABULAR COMPONENT

(76) Inventors: Paul Roberts, Monmouthshire (GB); Peter Grigoris, Athens (GR)

Correspondence Address: Leason Ellis LLP, 81 Main Street, Suite 503, White Plains, NY, 10601, US

(21) Appl. No.: 12/677,170

(22) PCT Filed: Sep. 11, 2008
(86) PCT No.: PCT/GB2008/003097

§ 371 Date: May 5, 2010

(30) Foreign Application Priority Data
Sep. 11, 2007 (GB) 0717692.8

(51) Int. Cl. A61F 2/32

(52) U.S. Cl. 623/22.36; 623/22.21

(57) ABSTRACT

An acetabular component (10) for hip surgery has an outer convex surface for location in the acetabulum and an inner concave bearing surface (16) suitable for holding and bearing against a corresponding femoral head component, without need for an interposed liner. The outer convex surface and the inner concave surface define between them an integral wall (14) of the component. Screw holes (18) are provided though the wall, from the inner concave bearing surface to the outer convex surface for screw fixation of the component in the acetabulum.

The present invention relates to acetabular components for use in hip surgery. The invention also relates to kits including such components and methods for implantation of such components.

### RELATED ART

In conventional hip replacement surgery, the replacement joint comprises a femoral part (which is intended to be inserted or implanted into the femur and which carries a ball-shaped articulation element) and an acetabular cup (which is intended to be inserted into the acetabulum). The acetabular cup is designed to hold a complementary cup-shaped liner (typically formed of plastics material), in which the ball-shaped element is to articulate.

One prior art document showing such an arrangement is WO 95/17140, in which the acetabular cup has a design including barb-shaped beads provided at the outer periphery to allow the cup to be pushed into a cavity formed in the acetabulum, these beads counteracting the removal of the cup from the cavity. As will be well understood, accidental removal of the cup from the cavity during service of the replacement hip is highly undesirable.

It is desirable for some patients to minimise the removal of bone from the original hip joint. This depends of course on the condition of the bone in the original hip joint. U.S. Pat. No. 4,123,806 proposes to minimise the removal of bone from the proximal femur by fitting a metallic hemispherical ball to a re-shaped head of the femur, rather than the conventional approach of amputating the whole head of the femur. In U.S. Pat. No. 4,123,806, a metallic acetabular cup is cemented into position in the acetabulum and a plastic hemispherical liner socket is located in the acetabular cup.

U.S. Pat. No. 4,801,300 discloses an acetabular cup formed of TiAl₆V₄. The cup has a flange ring having screw holes for screw fixing of the cup to the pelvis. This allows the cup to be fixed in position without the use of cement.

The range of ALLOFIT™ and ALLOFIT™-S cementless cups of Centerpulse (formerly Sulzer Orthopedics Ltd.) is disclosed in Lit. No. 2094e-Ed. 07-98 WWB, 2001. These products provide cementless implantable hemispherical cups having a metallic outer shell that, in some embodiments, has screw holes formed though the hemispherical wall of the shell, for screw fixing into the acetabulum. A plastic insert is then located into the metallic outer shell.

The range or TRILOGY™ Acetabular Systems of Zimmer, Inc. is disclosed in “Acetabular System: evolution through proven clinical experience” 97-6200-101 5ML, 1998. These products also provide metallic acetabular cups fitted with plastic liners. The cups are available with our without screw fixation holes formed though the hemispherical wall of the cups.

As the skilled person will appreciate, the prostheses mentioned above are merely representative of the different types of prosthesis available at the time of writing this disclosure.

It is known that the use of a metallic outer shell with a separate metallic liner results in a thick component which requires the removal of a significant amount of bone from the acetabulum. In order to avoid this, Centerpulse Orthopedics Ltd. (now Zimmer Inc.) provide DUROM™ Hip Resurfacing products, as disclosed in Lit. No. 06.01067.012-Ed. 03/2003 WL and Lit. No. 06.01068.012-Ed. 05/2003 WL. These products provide a monobloc metallic acetabular cup that has a porous outer hemispherical surface for long-term biological fixation with the acetabulum and peripheral circumferential fins for primary fixation in the acetabulum. The femoral component is also metallic and fits over the shaped femoral head with a minimum of bone loss. These components are formed from forged Co-28Cr-6Mo-0.2C/Protasul-21 WF (ISO 5832-12), a high carbon CoCr alloy. The femoral component fits directly inside the acetabular cup, providing metal-on-metal contact. As a consequence, the bearing surfaces must be very smooth, with precise control of sphericity and clearance and this is achieved by very careful micromachining processes. Such hip resurfacing operations are particularly suited to younger, more active patients.

### SUMMARY OF THE INVENTION

Conventional wisdom dictates that if adequate primary fixation for an uncemented acetabular cup cannot be achieved, due to deficiencies of the bone stock of the acetabulum, then additional fixation should be achieved using screws that are passed through holes in the shell, as described above. It is then necessary for a liner (which can be plastics (e.g. polyethylene, metallic or ceramic)) to be inserted into the acetabular cup to provide the bearing surface.

In contrast, acetabular components used in hip resurfacing operations should preferably be formed in one piece in order to minimise the thickness of the component. However, it has conventionally been considered that it would be highly undesirable for a hip resurfacing acetabular cup to be fixed in place using screws through holes in the bearing surface of the cup, due to the perceived risk to the lubrication and wear properties of the finished joint.

It has been proposed instead to use screw fixation for the acetabular component of a hip resurfacing system by providing a plate or flange to the acetabular component, though which screws can be fixed into the bone of the ilium (pelvis). However, such techniques can be technically demanding and may result in mal-positioning of the acetabular component, resulting in a risk of instability and/or accelerated wear of the bearing surface.

In another alternative configuration, small screws can be passed through the rim of the acetabular component, although this is technically difficult as in general the acetabular component is no greater than 4 mm thick at the rim.

In order to address one or more of the problems identified above, and preferably to reduce, ameliorate or even overcome said problems, the present inventors have realised that it is in fact possible to provide a suitable acetabular component that uses trans-articular screw fixation whilst still providing acceptable tribological properties for the component and also providing blocking of fluid transmission through the acetabular component.

Accordingly, in a first aspect, the present invention provides an acetabular component for hip surgery having an outer convex surface for location in the acetabulum and an inner concave bearing surface suitable for holding and bearing against a corresponding femoral head component, the outer convex surface and the inner concave surface defining between them an integral wall of the component, at least one screw hole being provided though the wall, from the inner concave bearing surface to the outer convex surface for screw fixation of the component in the acetabulum.

In this way, the present inventors provide a component that goes against a widely-held prejudice that it is not possible to provide screw holes in a bearing surface (also known as the articular surface) of an acetabular component, particular where the component is a monobloc providing metal-on-metal bearing. Furthermore, the omission of a bearing liner so that only an integral wall is provided between the inner bearing surface and the outer surface of the component means that the dimensions of the component can be limited to allow only minimal bone removal to accommodate the component in the acetabulum, which is a main aim of hip resurfacing surgery.

The outer surface of the acetabular component may have a porous or undulating surface finish, in order to provide a biocompatible surface for promoting bone ingrowth and hence suitable secondary fixation.

Preferably, the at least one screw hole is provided at a position surrounded by the inner bearing surface. In this way, the screw hole is provided located in a region of the bearing surface, rather than at an extremity of the bearing surface. For example, the inner concave bearing surface may be defined in terms of a rim and a point of maximum depth. It is possible then to define a distance along the inner concave bearing surface from the rim to the point of maximum depth. Preferably the least one screw hole is provided at a position ranging to 100% to 10% of said distance, when measured from the rim. Thus, the screw hole can be located anywhere in the bearing surface, except preferably not close to the rim. The lower limit of this range may be 15%, 20%, 25%, 30%, 35% or even 40%. The upper limit of the range may be lower than 100%, in which case the screw hole is not located at the point of maximum depth of the inner bearing surface. For example, the upper limit may be 95%, 90%, 85%, 80%, 75%, 70%, 65% or even 60%. These ranges may be combined in any combination.

From another perspective, the location of the screw hole can be considered in terms of angular position between the rim and the point of maximum depth of the bearing surface of the component, in which the rim is positioned at about 0° and the point of maximum depth is positioned at about 90°, measured about a point at the centre of the opening defined by the rim. This angle may be at least 10°, at least 15° or at least 20°. Preferably it is at most 80°, at most 70°, at most 60°, at most 50° or at most 40°. A preferred range is between 25° and 65°. Most preferably it is about 30°. The reason for this, as will be explained below, is that this allows the direction of the screw to be close to the average weight-bearing axis of the component, in use. By “close to” here, it is intended that the direction of the screw will be within about 15° of the weight being axis.

The component is preferably implanted in the acetabulum so that the screw hole (or screw holes) overlies a region of the ilio-pubic bar at which there is a good thickness of bone.

Preferably, the screw hole is directed so that, when a suitable screw is inserted through the hole and fixed in place, the screw lies perpendicular, or close to perpendicular, to the articular surface. As discussed below, this allows the more ready formation of a suitable sealing arrangement at the screw hole. It also allows more easy insertion of the screw by the surgeon.

Preferably, the at least one screw hole is provided with a screw thread for engagement with a corresponding screw thread located at a head of a fixation screw. This allows the screw head to be fixed in position with respect to the acetabular component. Of course, the main screw thread of the screw allows the screw to be fixed with respect to the acetabulum itself. Thus, the component is provided with a firm primary fixation to the acetabulum and the screw is substantially prevented from backing out from the screw hole during the lifetime of the component.

Preferably, the component is provided with at least one screw for location in the at least one screw hole. The at least one screw preferably has a distal shaft with a first screw thread and a proximal head with a second screw thread separate from the first screw thread, the at least one screw hole having a screw thread for engagement with the second thread.

The first screw thread may have a different pitch to the second screw thread. This means that the component must be carefully implanted in the patient, since the distance moved by the screw per turn with respect to the acetabulum and with respect to the acetabular component will be different. However, such arrangement provides firm fixation of the screw and hence of the acetabular component.

In an alternative arrangement, the distance moved by the screw per turn with respect to the acetabulum and with respect to the acetabular component is substantially the same. This requires that the pitch of the first screw thread is substantially the same as the pitch of the second screw thread. The pitch may be defined as the distance between corresponding points on adjacent parts of the same thread, measured along a line parallel to the principal axis of the screw. Typically, the first screw thread is the only thread on the distal shaft, i.e. the distal shaft is single-threaded. However, preferably the head has at least two threads, e.g. the second thread and optionally a third thread, interleave with each other. The second and third threads define, in effect, a multi-start thread for the head. The appearance of the head to the casual observer, therefore, will be that the pitch of the thread on the head is shorter than the pitch of the thread on the distal shaft. However, a closer inspection will show that the head actually has more interleaved threads than the distal shaft, but each having substantially the same pitch. This allows the head to be firmly fixed in the screw hole, even though the depth of the wall of the acetabular component may be small.

The screw hole in the acetabular component may be tapered. If this is the case, then preferably the head of the screw has a corresponding shape to provide a snug fit in the screw hole. If the taper angle is defined as the angle between a line following the peaks of the thread of the screw hole (in the same direction as the pitch of this thread) and the principal axis of the screw hole, preferably the taper angle is at least 1°. The taper angle may be 10° or less. A maximum angle for the taper may be lower, e.g. 9° or less, 8° or less, 7° or less, 6° or less, 5° or less, or most preferably 4° or less.

Alternatively, the screw head or sealing cap (see below) has a substantially cylindrical form (including screw thread(s) at its outer periphery) and has a substantially flat distal surface formed substantially perpendicular to the cylinder principal axis. This surface may form a suitable seal in use with a corresponding substantially flat surface formed at the screw hole in the component. A combination of cylindrical surfaces and flat distal surfaces for the screw head can be more straightforward to manufacture than the tapered structure set out above.

Preferably, an end surface of the head of the at least one screw, when said screw is located in the at least one screw hole, is located at a non-zero depth from the inner bearing surface of the component. Thus, the upper surface of the head of the screw may be recessed from the inner bearing surface. This means that it is possible to provide suitable screw driving means in the head of the screw (as is typical in surgical screws for bone repair such as transarticulation screws) whilst still providing suitable tribological characteristics to the component. The depth of the recess may be at least 0.1 mm. This depth may be 2 mm or less. Preferably, the depth is about 1 mm.

Preferably, the thickness of the wall of the acetabular component is 7 mm or less, 6 mm or less or 5 mm or less. A preferred thickness is about 4 mm.

Preferably, when said screw is located in the at least one screw hole, a sealing engagement is formed between said head and said screw hole. It is intended that the seal is perfected when the screw is located to the optimum extent in the screw hole, i.e. when the acetabular component is fixed in position in the acetabulum.

The reason for providing a seal is that the hydrostatic pressure in a hip joint can be very high during use. Typical hydrostatic pressures are considered to be up to about 1 bar in the hip joint of a typical human subject. The lubricating fluid in the joint is placed under pressure by the load bearing of the joint. This fluid is synovial fluid, considered to be similar to (or identical to) the fluid in a fully natural joint. If screw holes are provided in the bearing surface of the replacement joint, then there is a risk that this variable and often high hydrostatic pressure will be transmitted through the screw holes and into the acetabulum into which the screws are fixed. Corresponding flow of fluid will be expected. It is considered that this may lead to a risk of bone resorption in the acetabulum and consequently a reduced lifespan for the replacement joint.

It is not considered necessary that there is an absolute fluid-tight seal provided between the screw hole and the screw head. Thus, the present invention is intended to encompass arrangements that provide a substantial sealing at this location, since this can significantly reduce the transmission of hydrostatic pressure waves into the bone.

Additionally or alternatively it is possible to provide a seal between the head or shaft of the screw and the screw hole. For example, an O-ring seal may be provided, for example at the distal end of the screw hole and supported in the hole by a suitable flange. Additionally or alternatively, it is possible to provide a sealing cap, said sealing cap adapted to seal said screw hole after location of the screw. An O-ring seal may be provided at the screw head (or sealing cap), supported on said head (or cap) for sealing with the screw hole. Preferably, after location of the sealing cap in the hole, an end surface of said sealing cap is located at a non-zero depth from the inner bearing surface of the component. A sealing cap may be used to seal one or more screw holes that are provided in the acetabular component but which are not used to locate screws in the implantation operation. The sealing cap preferably has a similar or identical outer profile to the head of the screw. In effect, it can be considered for some embodiments of the present invention that the screw is provided with an integral sealing cap at the proximal end of the screw.

Preferably more than one screw hole is provided. For example, 2, 3 or 4 screw holes may be provided. Typically, these are provided in a cluster. For example, two or all of the screw holes may be provided in the same quadrant of the acetabular component. Preferably, in use, the cluster is aligned with the ilio-pubic bar of the patient.

The boundary between the bearing surface and the screw hole preferably has a radius of curvature of at least 0.2 mm. Preferably this radius of curvature is at most 2 mm.

A suitable material for the acetabular component is a cobalt-chrome alloy. The screw(s) and/or sealing cap(s) may be formed of the same material as the acetabular component.

The bearing surface of the acetabular component preferably has a surface roughness Ra of 0.05 μm or less, more preferably 0.04 μm or less, 0.03 μm or less, 0.02 μm or less, and most preferably 0.01 μm or less.

In a second aspect, the present invention provides a kit for hip surgery, including an acetabular component as set out with respect to the first aspect. Preferred optional additional elements for the kit include one or more screws as set out with respect to the first aspect, one or more sealing caps as set out with respect to the first aspect, and a femoral head component, adapted to slidingly articulate within the recess defined by the inner bearing surface of the acetabular component.

In a third aspect, the present invention provides an assembly of a prosthetic hip joint, including an acetabular component according to the first aspect and a femoral head component, a bearing surface of the femoral head component bearing directly against the inner bearing surface of the acetabular component, with an optional fluid lubricating film interposed between said bearing surfaces.

In a fourth aspect, the present invention provide a method for implantation of an acetabular component according to the first aspect in the acetabulum of a patient, the method including providing primary fixation of the acetabular component in the acetabulum by passing fixation screws through the screw holes provided in the inner bearing surface of the acetabular component.

Preferred and/or optional features of the invention are applicable either single or in any combination to any aspect of the invention, unless the context demands otherwise.

### BRIEF DESCRIPTION OF THE DRAWINGS

Preferred embodiments of the invention will now be described, by way of example, with reference to the accompanying drawings, in which:

FIG. 1 shows a schematic cross-sectional view of an acetabular component according to an embodiment of the invention, located in a preferred orientation for use.

FIG. 2 shows a plan view of the component of FIG. 1, viewed at its open side to show the bearing surface.

FIG. 3 shows a magnified cross-sectional view of a screw hole in the component of FIG. 1.

FIG. 4 shows a magnified cross-sectional view of an alternative screw hole in the component of FIG. 1.

FIG. 5 shows a schematic view of a screw suitable for use with the component of FIG. 1.

FIG. 6 shows a schematic view of a sealing cap suitable for use with the component of FIG. 1.

FIG. 7 shows a side view of a screw to be used in another embodiment of the invention.

FIG. 8 shows an isometric view of the screw of FIG. 7.

FIGS. 9 and 10 show front and rear perspective views of an acetabular component for use with screws according to FIGS. 7 and 8.

FIG. 11 shows a cross sectional view of the acetabular component of FIGS. 9 and 10.

FIG. 12 shows a front plan view of the acetabular component of FIGS. 9 and 10.

FIG. 13 shows a perspective view of a sealing cap for use in an embodiment of the invention.

FIG. 14 shows a cross sectional view of part of an acetabular component with the sealing cap of FIG. 13 fitted.

FIG. 15 shows a perspective view of a device for carrying out experimental tests in relation to the embodiments of the present invention.

FIGS. 16 and 17 show cross sectional and front plan views of the device of FIG. 15, respectively.

FIG. 18 shows an enlarged partial cross sectional view of the location of the sealing cap in the device of FIGS. 15-17.

FIG. 19 shows the pressure cycles used in dynamic pressure testing of the seal.

FIG. 20 shows an enlarged partial cross sectional view of the location of the sealing cap in a modified version of the device of FIGS. 15-17.

### DETAILED DESCRIPTION OF THE PREFERRED EMBODIMENTS

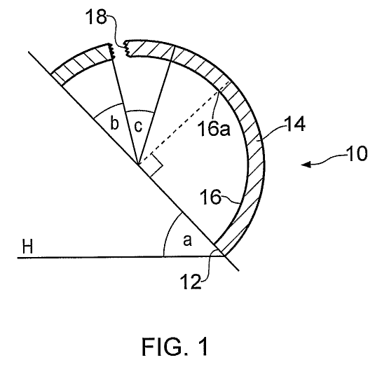

FIG. 1 shows a schematic cross-sectional view of an acetabular component 10 according to an embodiment of the invention, having a rim 12, a wall 14 and being formed to provide a substantially hemispherical concave bearing surface 16. The view of the component is taken along a plane coinciding with the diameter of the rim 12 and the point 16a of maximum depth of the inner bearing surface from the rim 12.

Screw hole 18 is provided through the thickness of wall 14.

The orientation of the component 10 to the horizontal line H is chosen to be such that angle a is about 45°. The location of screw hole 18 is chosen to be such that angle b is about 30°. It is intended that screw hole 18 (or a cluster of two or three screw holes) can be arranged in the area defined by angle c, which is also about 30°. The result of this is that the screw holes lie within about 15° of the vertical, substantially corresponding to the weight-bearing axis in use.

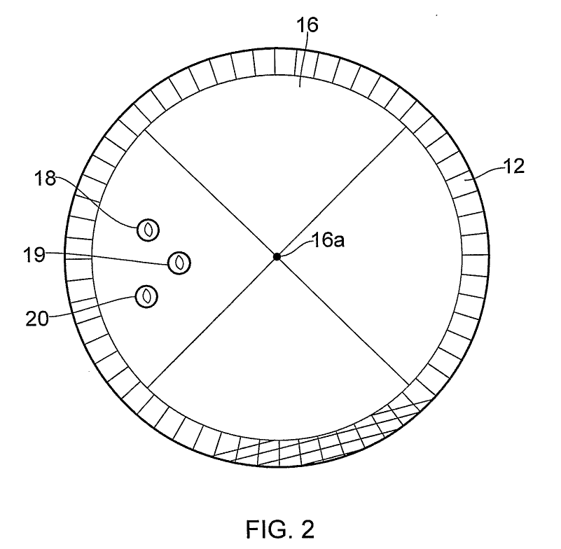

FIG. 2 shows a plan view of the component of FIG. 1, viewed at its open side to show the bearing surface 16 and a cluster of screw holes 18, 19, 20. The screw holes are clustered about the preferred optimum position between 30° and 60°. The bearing surface if notionally divided into four quadrants in the drawing. All of the screw holes are provided in one quadrant. The other quadrants do not contain screw holes. This provides a large area of bearing surface that is not affected by any possible deleterious local effects of the screw holes.

FIG. 3 shows a magnified cross-sectional view of a screw hole 18 in the component of FIG. 1. The screw hole 18 is formed through the thickness of wall 14, from the inner bearing surface 16 to the outer surface 17. The screw hole is threaded. The screw hole is formed with an inward taper, so that the screw hole becomes narrower as it progresses from the inner bearing surface 16 to the outer surface 17. The angle of the taper is defined as d in FIG. 3, which is the angle between the principal axis P of the screw hole and a line 22 which coincides with the peaks of the threads of the screw hole, in the direction of the pitch of the threads.

In FIG. 3, screw hole 18 has two interleaved threads, i.e. there are two threads with different circumferential start and end points. The pitch of these interleaved threads is identical, as shown by distances 24 and 26.

The boundary 28 between the screw hole 18 and the inner concave bearing surface 16 has a radius of curvature in the range 0.2-2 mm.

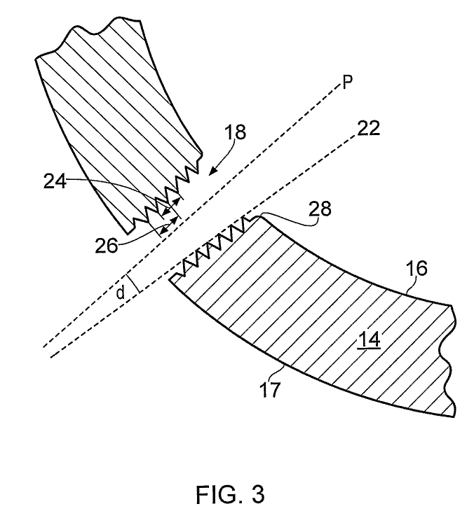

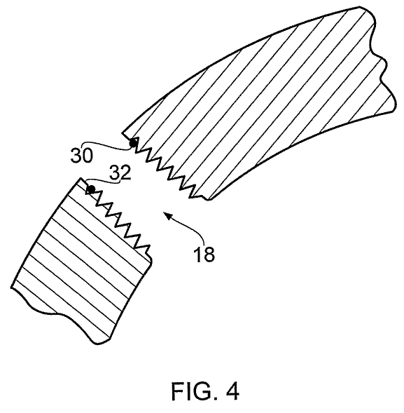

FIG. 4 shows an alternative embodiment to FIG. 3, in which an O-ring 30 is provided at a distal part of screw hole 18, supported by a flange 32. O-ring 30 is compressed against the head or shaft of the screw in use, thereby providing improved sealing.

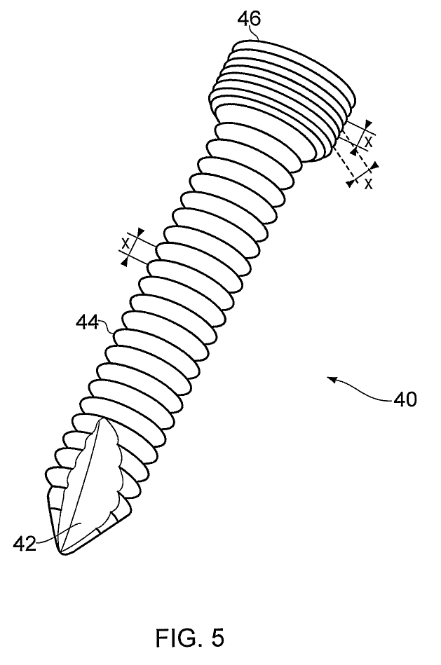

FIG. 5 shows a screw 40 for use in the present invention. This screw is a tip-cut screw, as evidenced by the cutting recess 42 shown at the tip, to provide a degree of self-tapping to the screw. Recess 42 allows for the collection of swarf from the self-tapping. The shaft 44 of the screw has a single thread of pitch X, wherein X is about 1 mm, or may be higher, e.g. about 2 mm. The head 46 is tapered to correspond with the shape of screw hole 18 and has two interleaved threads, also with pitch X. These two interleaved threads correspond to the threads formed in screw hole 18.

Although not shown, the drive head of the screw preferably has a hexagonal allen-style recess, as is well known in this field.

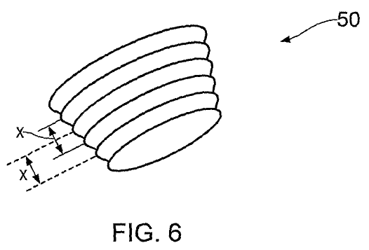

In use, it is intended that the acetabular component 10 is fixed in place using at least two screws. The remaining screw hole is sealed using a sealing cap 50, illustrated in FIG. 6. In effect, sealing cap 50 has an outer profile similar to that of head 46 of screw 40, in particular with respect to the thread and the drive head (not shown).

The material of all of the components is a forged chrome-cobalt alloy known for use in hip prostheses, such as Co-28Cr-6Mo-0.2C (ISO 5832-12). The surface finish is applied to the concave bearing surface by known finishing techniques.

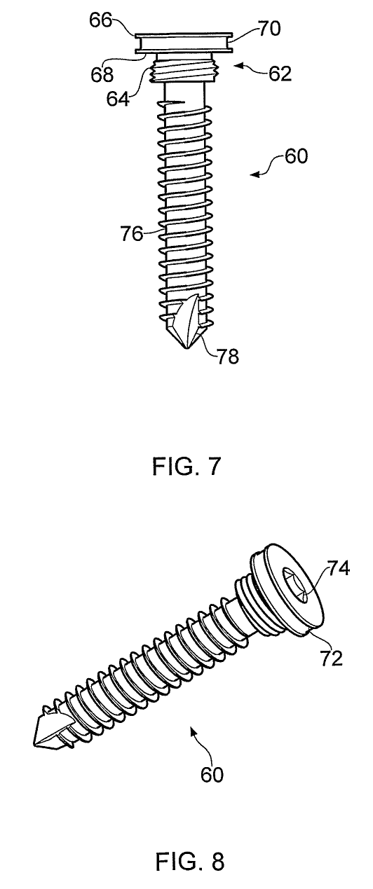

FIGS. 7 and 8 show a screw 60 for use in another embodiment of the invention. Screw 60 has a head 62 with a stepped substantially cylindrical shape of total axial length 3.6 mm. A first portion 64 of the head has a first diameter (equivalent to M5) and has a twin thread, each thread having a 1 mm pitch and the distance between adjacent threads being 0.5 mm The axial extend of the twin thread is 1.66 mm. A second portion 66 of the head has a second diameter (7.25 mm), larger than the first diameter. The second portion 66 is located proximally of the first portion. The step change in diameter between the first and second portions of the head provides a substantially flat surface 68 facing distally. The second portion of the head has an annular recess 70 formed at its outer circumferential surface for location of an O-ring 72 (not shown in FIG. 7 but shown in FIG. 8). The proximal surface of the head is provided with a hexagonal driving recess 74 of diameter 2.5 mm.

The main shank 76 of the screw 60 has an axial length 20 mm and is provided with a single thread of pitch 1 mm. Axial lengths of up to 50 mm are contemplated. The maximum diameter (corresponding to the peaks of the thread) of the main shank 76 is 4 mm, but greater diameters are contemplated. The minimum diameter (corresponding to the valleys of the thread) of the main shank 76 is 3 mm. The screw has a cutting tip 78 in order to allow the screw to be self-tapping.

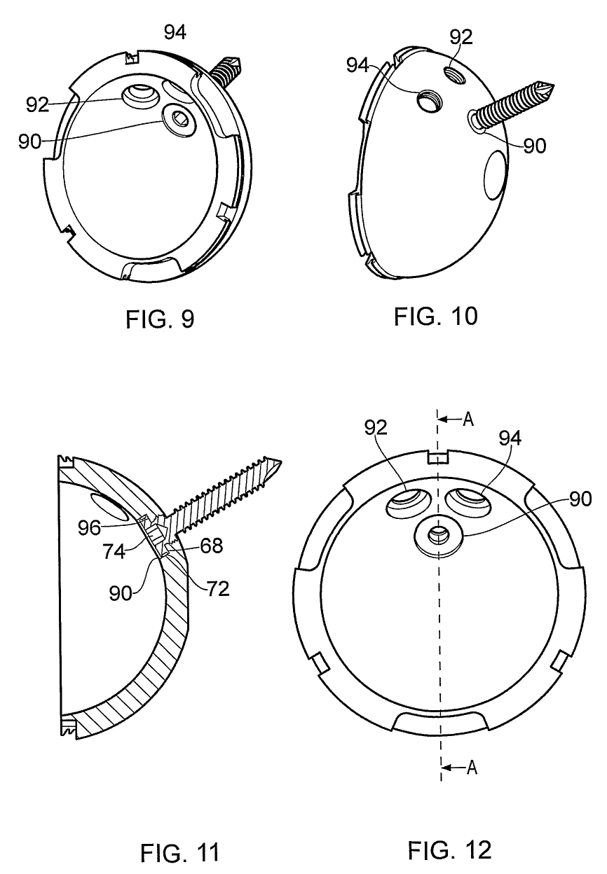

FIGS. 9 and 10 show views of an acetabular component for use with screws according to FIGS. 7 and 8. The component shown in these figures has an outer profile similar to the DUROM™ product mentioned above, specifically having equatorial fins and recesses for the cup holder. It is to be understood that these features may be preferred in the component, but the features considered to be of most significance in the present embodiment relate to the inner bearing surface of the component and the screw holes. Features similar to the embodiment of FIGS. 1 and 2 are not described again here. However, a significant difference between this embodiment and FIGS. 1 and 2 is the shape of the screw holes 90, 92, 94 in FIGS. 9 and 10.

FIG. 11 shows a cross sectional view of the same acetabular component, illustrating the seal that is provided by O-ring 72 and the inner circumferential surface of screw hole 90. Additionally, sealing is provided between distally-facing flat surface 68 of the screw head and corresponding shaped flat surface 96 of the screw hole. FIG. 12 shows a front plan view of the same acetabular component.

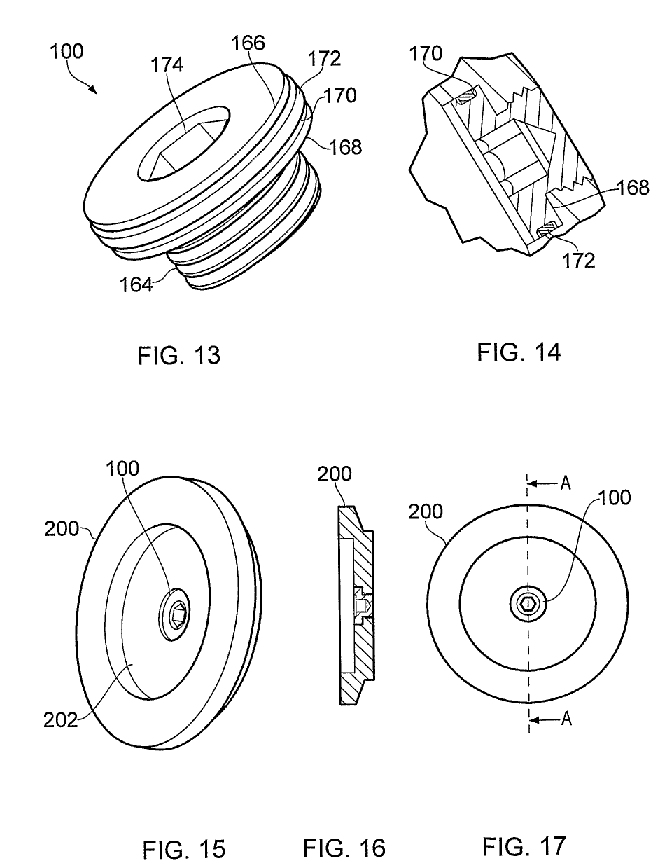

FIG. 13 shows a perspective view of a sealing cap 100 for use in an embodiment of the invention. FIG. 14 shows a cross sectional view of part of an acetabular component with the sealing cap 100 of FIG. 13 fitted. As will be clear, features of the sealing cap 100 are similar to features of the head 62 of screw 60 shown in FIG. 7. Sealing cap 100 has a stepped substantially cylindrical shape. A first portion 164 of the sealing cap has a first diameter with a twin thread. A second portion 166 of the sealing cap has a second diameter, larger than the first diameter. The second portion 166 is located proximally of the first portion 164. The step change in diameter between the first and second portions of the sealing cap provides a substantially flat surface 168 facing distally. The second portion of the sealing cap has an annular recess 170 formed at its outer circumferential surface for location of an O-ring 172. The proximal surface of the sealing cap is provided with a hexagonal driving recess 174.

As shown in FIG. 14, the proximal surface of the sealing cap, when the sealing cap is sealingly located in the screw hole, does not intersect the notional extension of the bearing surface of the acetabular component. Thus, there is a non-zero radial depth between where the bearing surface would be if there were no screw hole at that location, and the proximal surface of the sealing cap. Similar geometrical considerations apply to the proximal surface of the screw head, when a screw is fitted in the screw hole. In FIG. 14, it is indicated that the radius of curvature R for the bearing surface is 19 mm. This represents a relatively small radius of curvature for an acetabular component.

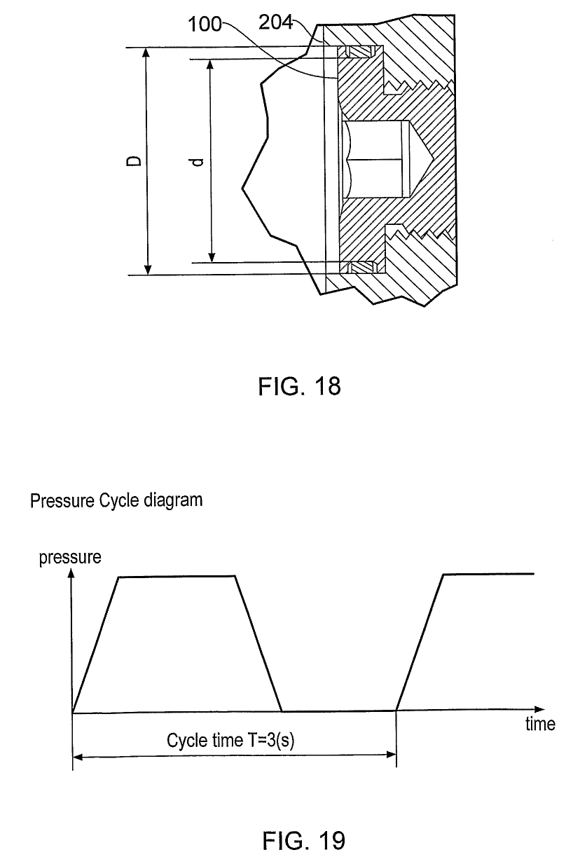

Sealing cap 100, formed of stainless steel, was used in a series of experiments to test whether a satisfactory seal could be obtained in practice. Sealing cap 100 was fitted into a disk-shaped seating 200 formed of stainless steel, as shown in FIGS. 15-17. Sealing cap 100 is shown here as having a recess at its outer periphery, the recess having outer diameter d for holding the O-ring. Seating 200 includes a central recess 202, at the centre of which is screw hole 204, of maximum diameter D. An enlarged partial cross sectional view of the location of the sealing cap in the screw hole 204 is shown in FIG. 18. It will be immediately understood that the seating 200 does not include a curved bearing surface, and so itself it not suitable as an acetabular component. However, the geometry of the screw hole 204 is identical to that used in the acetabular component, and so the combination of the seating 200 and the sealing cap 100 provides a suitable experimental arrangement for testing the sealing capability of the sealing cap and screw hole.

Testing was carried out to determine whether the function of the sealing screws and sealing caps is fulfilled. It is considered that, in use, the seal should withstand a maximum pressure of about 1 bar.

Two types of tests were performed: static and dynamic pressure tests. The sealing function was tested with the sealing caps described above located in the seating described above. The seating and sealing cap assembly was clamped to a sealed flanged cylindrical tube to form a watertight chamber. A tinted water solution was then used to half-fill the chamber. A cover with a connector for an air pressure supply was clamped to the top of the chamber (also sealed). The chamber was held vertically with a lab clamp and was set upon an absorbent filter to detect any leakage. Tests were carried out at room temperature (approx. 20° C.)

In the experiments, the following dimensions were used (refer to FIG. 18):
Measured seating diameter D=7.34 mm
Measured plug diameter d=6.45 mm
Standard O-ring dimensions: 6.3×0.6 mm (NBR Nitrile 70 Shore A)
In use, O-ring compression was about 25%

Tests were carried out under different pressure values and conditions. An absorbent filter was weighed (mg precision) before and after each test to be able to determine the amount of leakage if necessary.

The results of the static pressure tests were as shown in Table 1.

TABLE 1
Results of static pressure tests for FIG. 18

| Pressure (bar) | Time (hours) | Observations |
|---|---|---|
| 1 | 1 | no signs of leakage |
| 2.5 | 1.5 | no signs of leakage |
| 5 | 1.5 | no signs of leakage |
| 8 | 0.5 | no signs of leakage |

Dynamic pressure tests were also carried out, using an arrangement of pressure regulator, electro valves, flow control valves and pressure gauge in combination with the experimental arrangement for the static pressure tests.

Tests were carried out under different pressure values and conditions. The absorbent filter was observed after each test in order to detect any leakage of the tinted solution during the test. The pressure cycle time was set at 3 seconds, as illustrated in FIG. 19.

Table 2 shows the dynamic pressure test results

TABLE 2
Results of dynamic pressure tests for FIG. 18

| Pressure | Time | | |
|---|---|---|---|
| (bar) | (hours) | Pressure cycles | Observations |
| 1 | 15 | 18000 | no signs of leakage |
| 2.5 | 15 | 18000 | no signs of leakage |
| 5 | 15 | 18000 | no signs of leakage |

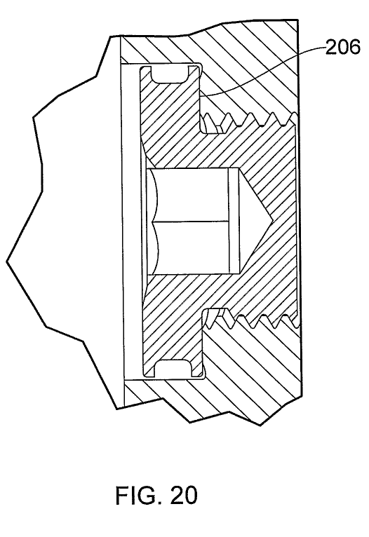

Corresponding testing was carried out using an identical arrangement but without an O-ring. Similar results were achieved, i.e. no leakage was observed under any of the conditions tested. FIG. 20 shows a view similar to that of FIG. 18 but without an O-ring. The sealing is provided by the abutment of the flat metal surfaces of the sealing cap and the screw hole, shown in FIG. 20 at interface 206.

Table 3 shows the results of static pressure testing for the arrangement of FIG. 20.

TABLE 3
Results of static pressure tests for FIG. 20

| Pressure (bar) | Time (hours) | Observations |
|---|---|---|
| 1 | 0.5 | no signs of leakage |
| 2.5 | 0.5 | no signs of leakage |
| 5 | 0.5 | no signs of leakage |

Note that in the tests reported, the sealing cap was tightened into the screw hole by hand only (no torque wrench was used).

Preferred embodiments of the invention have been described by way of example. Modifications of these embodiments, further embodiments and modifications thereof will be apparent to the skilled person on reading this disclosure and as such are within the scope of the invention.

1. An acetabular component for hip surgery comprising an outer convex surface for location in the acetabulum and an inner concave bearing surface suitable for holding and bearing against a corresponding femoral head component, the outer convex surface and the inner concave surface defining between them an integral wall of the component, at least one screw hole being provided though the wall, from the inner concave bearing surface to the outer convex surface for screw fixation of the component in the acetabulum.

2. An acetabular component according to claim 1 wherein said at least one screw hole is provided at a position surrounded by the inner bearing surface.

3. An acetabular component according to claim 2 wherein said at least one screw hole is provided with a screw thread for engagement with a corresponding screw thread located at a head of a fixation screw.

4. An acetabular component according to claim 1 further comprising at least one screw for location in the at least one screw hole.

5. An acetabular component according to claim 4 wherein said at least one screw has a shaft with a first screw thread and a head with a second screw thread separate from the first screw thread, the at least one screw hole having a screw thread for engagement with the second thread.

6. An acetabular component according to claim 5 wherein the first screw thread has the same pitch as the second screw thread.

7. An acetabular component according to claim 5 wherein an end surface of the head of the at least one screw, when said screw is located in the at least one screw hole, is located at a non-zero depth from the inner bearing surface of the component.

8. An acetabular component according to claim 5 wherein, when said screw is located in the at least one screw hole, a substantial sealing engagement is formed between said head and said screw hole.

9. An acetabular component according to claim 5 wherein a seal is provided between the head of the screw and the screw hole by a sealing cap.

10. An acetabular component according to claim 9 wherein said sealing cap is adapted to seal said screw hole after location of the screw, or in place of the screw.

11. An acetabular component according to claim 9 wherein, after location of the sealing cap in the hole, an end surface of said sealing cap is located at a non-zero depth from the inner bearing surface of the component.

12. An acetabular component according to claim 1, further comprising at least one screw so as to define a kit, the screw being for location though said at least one screw hole for fixation of the acetabular component into the acetabulum of a patient.

13. An acetabular component according to claim 12 wherein the at least one screw has a shaft with a first screw thread and a head with a second screw thread separate from the first screw thread, the at least one screw hole having a screw thread for engagement with the second thread.

14. An acetabular component according to claim 13 wherein the first screw thread has the same pitch as the second screw thread.

15. An acetabular component according to claim 13 wherein an end surface of the head of the at least one screw, when said screw is located in the at least one screw hole, is located at a non-zero depth from the inner bearing surface of the component.

16. An acetabular component according to claim 12 wherein, when said screw is located in the at least one screw hole, a sealing engagement is formed between said head and said screw hole.

17. An acetabular component according to claim 12 wherein a sealing cap is provided for said at least one screw, said sealing cap adapted to seal said screw hole after location of the screw, and after location of the sealing cap in said hole, an end surface of said sealing cap being located at a non-zero depth from the inner bearing surface of the component.

18. An assembly of a prosthetic hip joint, comprising an acetabular component according to claim 1 and a femoral head component, a bearing surface of the femoral head component bearing directly against the inner bearing surface of the acetabular component, with an optional fluid lubricating film interposed between said bearing surfaces.

19. A method for implantation of an acetabular component according to claim 1, the method including providing primary fixation of the acetabular component in the acetabulum by passing fixation screws through the screw holes provided in the inner bearing surface of the acetabular component.
